(19) United States
(12) Patent Application Publication
(10) Pub. No.: US 2026/0207274 A1
(43) Pub. Date: Jul. 23, 2026

(54) TRANSLATIONAL LOCKING OF AN OUT-OF-VIEW CONTROL POINT IN A COMPUTER-ASSISTED SYSTEM

(71) Applicant: INTUITIVE SURGICAL OPERATIONS, INC., Sunnyvale, CA (US)

(72) Inventors: Thomas N. MCNAMARA, Los Gatos, CA (US); Nitin SHANKAR, Sunnyvale, CA (US); Jordan WONG, Burlingame, CA (US); Jonathan YUEN, San Jose, CA (US)

(21) Appl. No.: 19/140,489

(22) PCT Filed: Jan. 4, 2024
(86) PCT No.: PCT/US2024/010331

§ 371 Date: Jun. 18, 2025

Related U.S. Application Data
(60) Provisional application No. 63/478,565, filed on Jan. 5, 2023.

(51) Int. Cl. A61B 34/30; A61B 34/00; A61B 90/00

(52) U.S. Cl. CPC A61B 34/30; A61B 34/76; A61B 90/361; A61B 2034/305

(57) ABSTRACT

A computer-assisted device includes a repositionable structure that includes one or more joints coupled to an end effector; and a control unit, wherein the control unit is configured to: determine that a control point associated with the end effector is not within a field of view of an imaging device capturing images of a worksite; while the control point is not within the field of view of the imaging device, receive an actuation command for causing the control point to translate; based on the actuation command, generate a modified command that does not translate the control point; and actuate one or more joints of the repositionable structure or the end effector based on the modified command.

### RELATED APPLICATIONS

This application claims the benefit of U.S. Provisional Application No. 63/478,565, filed Jan. 5, 2023, and titled “TRANSLATIONAL LOCKING OF AN OUT-OF-VIEW CONTROL POINT IN A COMPUTER-ASSISTED SYSTEM,” which is incorporated by reference herein.

### TECHNICAL FIELD

The present disclosure relates generally to operation of computer-assisted systems with repositionable structures, such as articulated arms, and more particularly to limiting translational movement of portions of such a repositionable structure that are not within a field of view of an imaging device.

### BACKGROUND

Computer-assisted electronic systems are being used more and more often. This is especially true in industrial, entertainment, educational, and other settings. As a medical example, the medical facilities of today have large arrays of electronic systems being found in operating rooms, interventional suites, intensive care wards, emergency rooms, and/or the like. Many of these electronic systems may be capable of autonomous or semi-autonomous motion. It is also known for personnel to control the motion and/or operation of electronic systems using one or more input devices located at a user control system. As a specific example, minimally invasive, robotic telesurgical systems permit surgeons to operate on patients from bedside or remote locations. Telesurgery refers generally to surgery performed using surgical systems where the surgeon uses some form of remote control, such as a servomechanism, to manipulate surgical instrument movements rather than directly holding and moving the instruments by hand.

When a computer-assisted system is used to perform a task at a worksite (e.g., an interior anatomy of a patient in a medical example), one or more instruments of the computer-assisted system are positioned within a workspace that is created, for example, by insufflation of a gas into a region of the patient anatomy that surrounds the worksite. An imaging device, such as an endoscope, is typically inserted into the workspace. The imaging device is positioned and orientated so that relevant portions of the one or more instruments are within a field of view of the imaging device. This allows an operator of the computer-assisted system to observe and monitor the one or more instruments as a procedure is performed in the workspace. Thus, coordinated use of the imaging device and the one or more instruments is important.

Accordingly, improved techniques for controlling motion of instruments of a computer-assisted system that are being observed using an imaging device are desirable.

### SUMMARY

Consistent with some embodiments, a computer-assisted system comprises a repositionable structure that includes one or more joints coupled to an end effector and a control unit. In some embodiments, the control unit, when coupled to the repositionable structure, is configured to determine that a control point associated with the end effector is not within a field of view of an imaging device capturing images of a worksite, while the control point is not within the field of view of the imaging device, receive an actuation command for causing the control point to translate a specified distance, based on the actuation command, generate a modified command that prevents the control point from translating the specified distance, and actuate one or more joints of the repositionable structure or the end effector based on the modified command.

Consistent with some embodiments, a method for operating a computer-assisted device that includes a repositionable structure that includes one or more joints coupled to an end effector comprises determining, by a control unit, that a control point associated with the end effector is not within a field of view of an imaging device capturing images of a worksite; while the control point is not within the field of view of the imaging device, receiving, by the control unit, an actuation command for causing the control point to translate a specified distance; based on the actuation command, generating, by the control unit, a modified command that prevents the control point from translating the specified distance; and actuating, by the control unit using one or more motors, solenoids, servos, or actuators, one or more joints of the repositionable structure or the end effector based on the modified command.

In some embodiments a non-transitory machine-readable medium comprises a plurality of machine-readable instructions which when executed by one or more processors associated with a computer-assisted device are adapted to cause the one or more processors to perform the methods disclosed herein.

In some instances, the ability of an imaging device to assist an operator when controlling one or more instruments in a workspace is limited. For example, longer instruments often extend out of the field of view of the imaging device, particularly when the operator, such as a surgeon in a medical example, zooms in the field of view of the imaging device to confirm that a material has been captured correctly and/or a specific instrument is precisely located in the appropriate position and with the appropriate orientation. When the viewed parts of an instrument are commanded to move by the operator in such a situation, translation or other motion of unviewed parts of the instrument often occur to effect the motions commanded by the operator. Because the unviewed parts of the instrument are moving outside the field of view of the imaging device, it is not possible for the operator to fully monitor their movement. In such situations, it is helpful to alter actuation commands for the one or more instruments to restrict motions of portions of the one or more instruments that are not within a field of view of the imaging device.

The foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

FIG. 1 is a simplified diagram of a computer-assisted system according to some embodiments.

FIG. 2 is a simplified diagram showing shows a side view of an imaging device and an end effector of a computer-assisted system in a workspace according to some embodiments.

FIG. 3 is a simplified diagram showing an instrument, an end effector, and an associated articulated wrist joint according to some embodiments.

FIG. 4 is a simplified perspective diagram of the distal end of the end effector and articulated wrist joint of FIG. 2 according to some embodiments.

FIG. 5 is a simplified diagram showing a portion of an end effector extending outside of a field of view of an imaging device according to some embodiments.

FIG. 6 is a simplified diagram showing an end effector disposed within a field of view and coupled to control points outside of the field of view according to some embodiments.

FIG. 7 is a simplified diagram of an exemplary method for translational locking of out-of-view control points according to some embodiments.

In the figures, elements having the same designations have the same or similar functions.

### DETAILED DESCRIPTION

This description and the accompanying drawings that illustrate inventive aspects, embodiments, or modules should not be taken as limiting-the claims define the protected invention. Various mechanical, compositional, structural, electrical, and operational changes may be made without departing from the spirit and scope of this description and the claims. In some instances, well-known circuits, structures, or techniques have not been shown or described in detail in order not to obscure the invention. Like numbers in two or more figures represent the same or similar elements.

In the following description, specific details are set forth describing some embodiments consistent with the present disclosure. It will be apparent to one skilled in the art, however, that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional. The term “including” means including but not limited to, and each of the one or more individual items included should be considered optional unless otherwise stated. Similarly, the term “can” indicates that an item is optional.

Further, the terminology in this description is not intended to limit the invention. For example, spatially relative terms-such as “beneath”, “below”, “lower”, “above”, “upper”, “proximal”, “distal”, and the like-may be used to describe one element's or feature's relationship to another element or feature as illustrated in the figures. These spatially relative terms are intended to encompass different positions (i.e., locations) and orientations (i.e., rotational placements) of the elements or their operation in addition to the position and orientation shown in the figures. For example, if the content of one of the figures is turned over, elements described as “below” or “beneath” other elements or features would then be “above” or “over” the other elements or features. Thus, the exemplary term “below” can encompass both positions and orientations of above and below. A device may be otherwise oriented (rotated 90 degrees or at other orientations) and the spatially relative descriptors used herein interpreted accordingly. Likewise, descriptions of movement along and around various axes include various special element positions and orientations. In addition, the singular forms “a”, “an”, and “the” are intended to include the plural forms as well, unless the context indicates otherwise. And, the terms “comprises”, “comprising”, “includes”, and the like specify the presence of stated features, steps, operations, elements, and/or components but do not preclude the presence or addition of one or more other features, steps, operations, elements, components, and/or groups. Components described as coupled may be electrically or mechanically directly coupled, or they may be indirectly coupled via one or more intermediate components.

Elements described in detail with reference to one embodiment, embodiment, or module may, whenever practical, be included in other embodiments, embodiments, or modules in which they are not specifically shown or described. For example, if an element is described in detail with reference to one embodiment and is not described with reference to a second embodiment, the element may nevertheless be claimed as included in the second embodiment. Thus, to avoid unnecessary repetition in the following description, one or more elements shown and described in association with one embodiment, embodiment, or application may be incorporated into other embodiments, embodiments, or aspects unless specifically described otherwise, unless the one or more elements would make an embodiment or embodiment non-functional, or unless two or more of the elements provide conflicting functions.

In some instances, well known methods, procedures, components, and circuits have not been described in detail so as not to unnecessarily obscure aspects of the embodiments.

This disclosure describes various elements (such as systems and devices, and portions of systems and devices) with examples in three-dimensional space. In such examples, the term “position” refers to the location of an element or a portion of an element in a three-dimensional space (e.g., three degrees of translational freedom along Cartesian x-, y-, and z-coordinates). Also in such examples, the term “orientation” refers to the rotational placement of an element or a portion of an element (three degrees of rotational freedom-e.g., roll, pitch, and yaw). Other examples may encompass other dimensional spaces, such as two-dimensional spaces. As used herein, the term “pose” refers to the position, the orientation, or the position and the orientation combined, of an element or a portion of an element. As used herein, and for an element or portion of an element of a structure or assembly (e.g., of a computer-assisted system or a repositionable structure, etc.), the term “proximal” in a kinematic series refers to a direction toward the base of the kinematic series, and the term “distal” refers to a direction away from the base along the kinematic series.

Aspects of this disclosure are described in reference to electronic systems, computer-assisted devices, and robotic devices, which may include systems and devices that are teleoperated, remote-controlled, autonomous, semiautonomous, manually manipulated, and/or the like. Example computer-assisted systems include those that comprise robots or robotic devices. Further, aspects of this disclosure are described in terms of an embodiment using a medical system, such as the da Vinci® Surgical System commercialized by Intuitive Surgical, Inc. of Sunnyvale, California. Knowledgeable persons will understand, however, that inventive aspects disclosed herein may be embodied and implemented in various ways, including robotic and, if applicable, non-robotic embodiments. Embodiments described for da Vinci® Surgical Systems are merely exemplary, and are not to be considered as limiting the scope of the inventive aspects disclosed herein. For example, techniques described with reference to surgical instruments and surgical methods may be used in other contexts. Thus, the instruments, systems, and methods described herein may be used for humans, animals, portions of human or animal anatomy, industrial systems, general robotic, or teleoperational systems. As further examples, the instruments, systems, and methods described herein may be used for non-medical purposes including industrial uses, general robotic uses, sensing or manipulating non-tissue work pieces, cosmetic improvements, imaging of human or animal anatomy, gathering data from human or animal anatomy, setting up or taking down systems, training medical or non-medical personnel, and/or the like. Additional example applications include use for procedures on tissue removed from human or animal anatomies (with or without return to a human or animal anatomy) and for procedures on human or animal cadavers. Further, these techniques can also be used for medical treatment or diagnosis procedures that include, or do not include, surgical aspects.

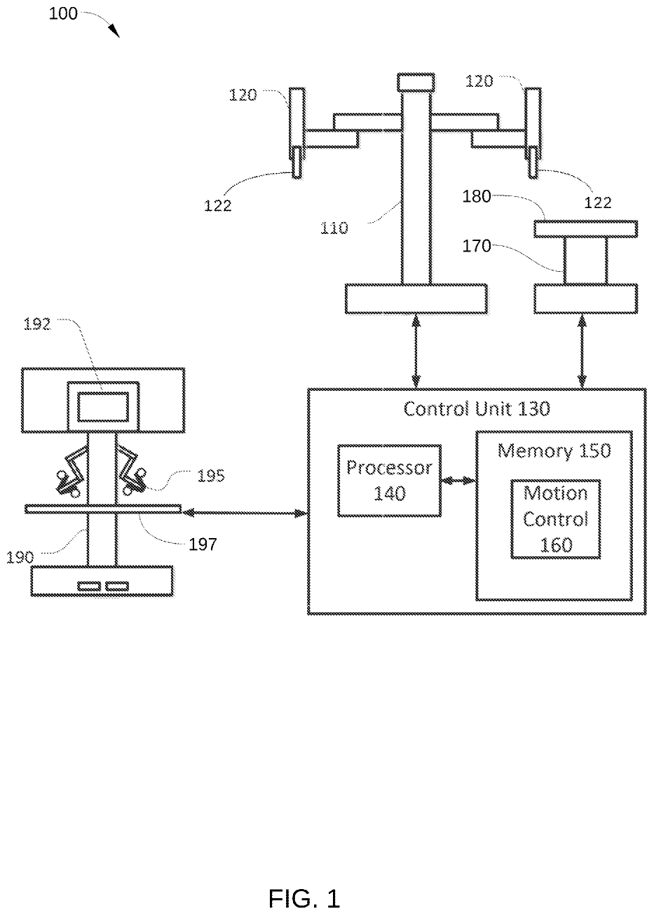

FIG. 1 is a simplified diagram of a computer-assisted system 100 according to some embodiments. As shown in FIG. 1, computer-assisted system 100 includes, without limitation, a device 110 with one or more movable or articulated arms 120. Each of the one or more articulated arms 120 is a repositionable structure that supports one or more instruments or end effectors 122. In some examples, device 110 is consistent with a computer-assisted surgical device. The one or more articulated arms 120 provide support for one or more instruments, surgical instruments, imaging devices, and/or the like mounted to a distal end of at least one of the articulated arms 120. Device 110 can further be coupled to an operator workstation 190, which can include one or more master controls for operating the device 110, the one or more articulated arms 120, and/or the end effectors. In some embodiments, device 110 and the operator workstation correspond to a da Vinci® Surgical System commercialized by Intuitive Surgical, Inc. of Sunnyvale, California. In some embodiments, computer-assisted surgical devices with other configurations, fewer or more articulated arms, and/or the like are optionally used with computer-assisted system 100.

Device 110 is coupled to a control unit 130 via an interface. The interface can include one or more wireless links, cables, connectors, and/or buses and can further include one or more networks with one or more network switching and/or routing devices. Control unit 130 includes, without limitation, a processor 140 coupled to memory 150. Operation of control unit 130 is controlled by processor 140. Although control unit 130 is shown with only one processor 140, it is understood that processor 140 can be representative of one or more central processing units, multi-core processors, microprocessors, microcontrollers, digital signal processors, field programmable gate arrays (FPGAs), application specific integrated circuits (ASICs), and/or the like in control unit 130. Control unit 130 can be implemented as a stand-alone subsystem and/or board added to a computing device or as a virtual machine. In some embodiments, control unit is included as part of operator workstation 190 and/or operated separately from, but in coordination with operator workstation 190. Some examples of control units, such as control unit 130, include non-transient, tangible, machine readable media that include executable code that, when run by one or more processors (e.g., processor 140), cause the one or more processors to perform the processes of method 700.

Memory 150 is used to store software executed by control unit 130 and/or one or more data structures used during operation of control unit 130. Memory 150 can include one or more types of machine-readable media. Some common forms of machine-readable media can include floppy disk, flexible disk, hard disk, magnetic tape, any other magnetic medium, CD-ROM, any other optical medium, RAM, PROM, EPROM, FLASH-EPROM, any other memory chip or cartridge, and/or any other medium from which a processor or computer is adapted to read.

As shown, memory 150 includes, without limitation, a motion control application 160 that supports autonomous and/or semiautonomous control of device 110. Motion control application 160 can include one or more application programming interfaces (APIs) for receiving position, motion, and/or other sensor information from device 110, exchanging position, motion, and/or collision avoidance information with other control units regarding other devices, such as a surgical table and/or imaging device, and/or planning and/or assisting in the planning of motion for device 110, articulated arms 120, and/or end effectors 122 of device 110. Although motion control application 160 is depicted as a software application, motion control application 160 can be implemented using hardware, software, and/or a combination of hardware and software.

Although the example of computer-assisted system 100 illustrated in FIG. 1 includes only one device 110 with two articulated arms 120, one of ordinary skill would understand that computer-assisted system 100 can include any number of devices with articulated arms and/or end effectors of similar and/or different design from device 110. In some examples, each of the devices can include fewer or more articulated arms and/or end effectors.

Computer-assisted system 100 further includes a surgical table 170. Like the one or more articulated arms 120, surgical table 170 supports articulated movement of a table top 180 relative to a base of surgical table 170. In some examples, the articulated movement of table top 180 includes support for changing a height, a tilt, a slide, a Trendelenburg orientation, and/or the like of table top 180. Although not shown, surgical table 170 can include one or more control inputs, such as a surgical table command unit for controlling the position and/or orientation of table top 180.

Surgical table 170 is also coupled to control unit 130 via a corresponding interface. The interface can include one or more wireless links, cables, connectors, and/or buses and can further include one or more networks with one or more network switching and/or routing devices. In some embodiments, surgical table 170 can be coupled to a different control unit than control unit 130.

Control unit 130 can further be coupled to an operator workstation 190 via the interface. Operator workstation 190 can be used by an operator, such as a surgeon, to control the movement and/or operation of the articulated arms 120 and end effectors 122. To support operation of the articulated arms 120 and end effectors 122, operator workstation 190 includes, without limitation, a display system 192 for displaying images of at least portions of one or more of the articulated arms 120 and/or end effectors. For example, display system 192 can be used when it is impractical and/or impossible for the operator to see articulated arms 120 and/or end effectors 122 as they are being used. In some embodiments, display system 192 displays a video image from a video capturing device, such as an endoscope, which is controlled by one of the articulated arms 120, or a third articulated arm (not shown).

Operator workstation 190 can further include a console workspace with one or more input controls 195 (or “master controls 195”) that can be used for operating device 110, articulated arms 120, and/or end effectors 122. Each of input controls 195 can be coupled to the distal end of an associated articulated arm 120 so that movements of input controls 195 can be detected by the operator workstation 190 and communicated to control unit 130. To provide improved ergonomics, the console workspace can also include one or more rests, such as an arm rest 197 on which operators can rest their arms while manipulating input controls 195. In some examples, display system 192 and input controls 195 can be used by the operator to teleoperate articulated arms 120 and/or end effectors 122 mounted on articulated arms 120. In some examples, input controls 195 include any type of device manually operable by a human user, e.g., joysticks, trackballs, button clusters, and/or other types of haptic devices typically equipped with multiple degrees of freedom. Position, force, and/or tactile feedback devices (not shown) can be employed to transmit position, force, and/or tactile sensations from the instruments back to the hands of the operator through input controls 195. In some embodiments, device 110, operator workstation 190, and control unit 130 correspond to a da Vinci® Surgical System commercialized by Intuitive Surgical, Inc. of Sunnyvale, California.

In some embodiments, other configurations and/or architectures are used with computer-assisted system 100. In some examples, control unit 130 is included as part of operator workstation 190 and/or device 110. In some embodiments, computer-assisted system 100 is found in an operating room and/or an interventional suite. In some embodiments, there are additional workstations 190 to control additional arms that can be attached to device 110. Additionally, in some embodiments, workstation 190 can have controls for controlling surgical table 170.

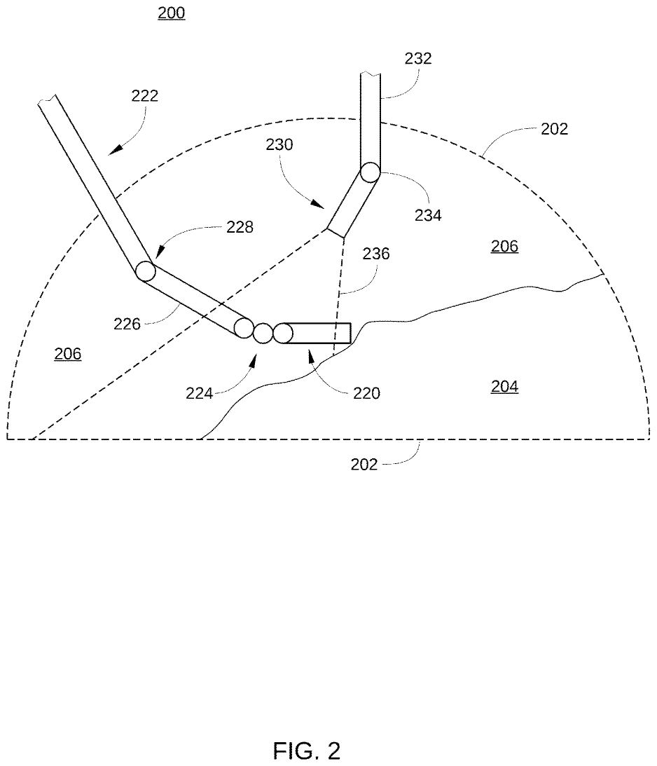

FIG. 2 is a simplified diagram showing shows a side view of an end effector 220 and an imaging device 230 of a computer-assisted system 200 in a worksite 202 according to some embodiments. For example, computer-assisted system 200 can be consistent with computer-assisted system 100 of FIG. 1. Worksite 202 indicates a region in which one or more end effectors 220 perform various tasks on a material 204, for example based on operator inputs via workstation 190 in FIG. 1. In a medical example, worksite 202 material 204 is a portion of internal patient anatomy and a cavity 206 that is created, for example, by insufflation of a gas into a region that surrounds internal patient anatomy 204. In the embodiment illustrated in FIG. 1, end effector 220 and imaging device 230 are positioned within cavity 206, although in other embodiments additional end effectors 220 and/or imaging devices 230 can be positioned within cavity 206.

Imaging device 230 can be any camera or optical device that can be mounted on an articulated arm 232 of computer-assisted system 200 and employed in worksite 202. For example, in some embodiments, imaging device 230 can include an endoscopic camera or other minimally invasive surgical imaging device that has a field of view 236 within worksite 202. In the example illustrated in FIG. 2, field of view 236 is depicted two-dimensionally as a triangular region within worksite 202, but in practice field of view 236 is typically a three-dimensional region, such as a pyramid, cone, or frustum. In some embodiments, imaging device 230 is coupled to articulated arm 232 via a multi-axis wrist joint 234 that enables orientation of imaging device 230 within worksite 202 in multiple directions. Thus, in such embodiments, imaging device 230 can be employed to provide direct visual observation of end effector 220 and worksite 202, such as material 204 (e.g., internal patient anatomy 204 in a medical example) and/or objects in worksite 202 while end effector 220 performs various tasks within worksite 202. In other embodiments, imaging device 230 can be coupled to articulated arm 232 with any technically feasible configuration of joints and links other than that shown in FIGS. 1 and 2.

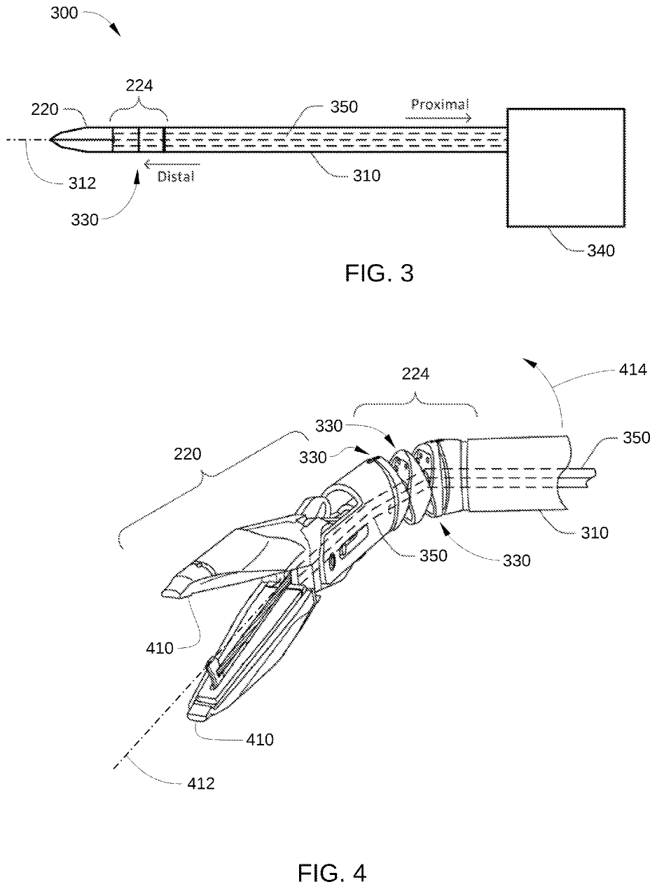

End effector 220 can be any instrument, tool, or other device that can be mounted on an articulated arm 222 of computer-assisted system 200 and employed in worksite 202. For example, in some embodiments, end effector 220 can include a specific minimally invasive surgical instrument, such as a surgical stapler, a suction irrigator, an electrocautery device for delivering energy, a gripper, a cutting mechanism, and/or the like. In such embodiments, end effector 220 is employed to perform one or more operations on material 204. In some embodiments, end effector 220 is coupled to articulated arm 222 via an articulated joint, such as an articulated wrist 224. In the embodiment illustrated in FIG. 1, end effector 220 is coupled to a link 226 via articulated wrist 224, and link 226 is coupled via a joint 228 to another link of articulated arm 222 that extends out of worksite 202. In other embodiments, end effector 220 can be coupled to articulated arm 222 with any other technically feasible configuration of joints and links. One embodiment of end effector 220 and articulated wrist 224 is described below in conjunction with FIGS. 3 and 4.

FIG. 3 is a simplified diagram showing an instrument 300 including end effector 220 and articulated wrist 224 according to some embodiments. The directions “proximal” and “distal” as depicted in FIG. 3 and as used herein help describe the relative orientation and location of components of end effector 220. Distal generally refers to elements in a direction further along a kinematic chain from a base of computer-assisted system 200, such as computer-assisted device 110 in FIG. 1, and/or closest to a worksite in the intended operational use of end effector 220. Proximal generally refers to elements in a direction closer along a kinematic chain toward the base of computer-assisted system 200 and/or one of the articulated arms of computer-assisted system 200.

As shown in FIG. 3, instrument 300 includes, without limitation, end effector 220 and articulated wrist 224, which couples end effector 220 to a long shaft 310. Thus, shaft 310 couples end effector 220 and articulated wrist 224 at a distal end of shaft 310 to an articulated arm and/or a computer-assisted device at a proximal end of shaft 310, such as a drive system 340. Depending on the particular procedure for which end effector 220 is being used, shaft 310 can be inserted through an opening (e.g., a body wall incision, a natural orifice, and/or the like) in order to place end effector 220 in proximity to a remote surgical site located within the anatomy of a patient, such as worksite 202 in FIG. 2. In the embodiment illustrated in FIG. 2, end effector 220 is generally consistent with a two-jawed gripper-style end effector. However, one of ordinary skill would understand that end effector 220 can be configured as any other suitable tool, device, surgical instrument, and the like that can be employed by computer-assisted system 200.

In some embodiments, end effector 220 relies on multiple degrees of freedom (DOFs) during operation. Depending upon the configuration of end effector 220, articulated arm 222, and/or the specific drive system 340 to which end effector 220 is coupled, various DOFs for positioning, orienting, and/or operating end effector 220 are possible. In some examples, shaft 310 is inserted in a distal direction and/or retreated in a proximal direction to provide an insertion DOF that is used to control how deep within the anatomy of the patient end effector 220 is positioned. In some examples, shaft 310 is able to rotate about a longitudinal axis 312 to provide a roll DOF that is used to rotate end effector 220. In some examples, additional flexibility in the position and/or orientation of end effector 220 is provided by articulated wrist 224, which is used to couple end effector 220 to the distal end of shaft 310. In some examples, articulated wrist 224 includes one or more rotational joints 330, such as one or more roll, pitch, or yaw joints that provide one or more “roll,” “pitch,” and “yaw” DOF(s), respectively. In such examples, such rotational joints 330 can be used to control an orientation of end effector 220 relative to the longitudinal axis of shaft 310. In some examples, the one or more rotational joints include a pitch and a yaw joint; a roll, a pitch, and a yaw joint, a roll, a pitch, and a roll joint; and/or the like. In some examples, end effector 220 can further include a grip DOF used to control the opening and closing of the jaws of end effector 220 and/or an activation DOF used to control the extension, retraction, and/or operation of a cutting mechanism or stapling mechanism included in end effector 220.

Generally, the drive system 340 associated with end effector 220 is located at the proximal end of shaft 310. Drive system 340 includes one or more components for introducing forces and/or torques to end effector 220 that can be used to manipulate the above-described DOFs supported by end effector 220. In some examples, drive system 340 includes one or more motors, solenoids, servos, active actuators, hydraulics, pneumatics, and/or the like that are operated based on signals received from a control unit, such as control unit 130 of FIG. 1. In some examples, the signals include one or more currents, voltages, pulse-width modulated wave forms, and/or the like. In some examples, drive system 340 includes one or more shafts, gears, pulleys, rods, bands, and/or the like which are coupled to corresponding motors, solenoids, servos, active actuators, hydraulics, pneumatics, and/or the like that are part of articulated arm 222, to which end effector 220 is mounted. In some examples, the one or more drive mechanisms 350, such as disks, shafts, gears, pulleys, rods, bands, and/or the like, are used to receive forces and/or torques from the motors, solenoids, servos, active actuators, hydraulics, pneumatics, and/or the like and apply those forces and/or torques to adjust the various DOFs of end effector 220. In some examples, shaft 310 is hollow and various drive mechanisms 350 pass along the inside of shaft 310 from drive system 340 to the corresponding DOF in end effector 220 and/or articulated wrist 224.

FIG. 4 is a simplified perspective diagram of end effector 220 and articulated wrist 224 according to some embodiments. In FIG. 4, the distal end of end effector 220 is depicted so that additional details of end effector 220, articulated wrist 224, and drive mechanisms 350 are visible. In more detail, end effector 220 includes opposing jaws 410 shown in an open position. Jaws 410 are configured to move between open and closed positions so that end effector 220 used during a procedure to grip and release tissue and/or other structures, such as sutures, located at the surgical site. Alternatively, in some examples, end effector 220 is configured as a surgical stapler, and jaws 410 are configured to move between open and closed positions so that end effector 220 can be used install one or more surgical staples. In some examples, jaws 410 are operated together as a single unit with both jaws 410 opening and/or closing at the same time. In some examples, jaws 410 can be opened and/or closed independently so that, for example, one jaw 410 is held steady while the other jaw 410 is opened and/or closed.

In some examples, a commanded motion of one control point included in articulated arm 222, such as a distal portion or tip of a jaw 410 of end effector 220, is effectuated by rotation and/or translation of one or more different control points included in articulated arm 222, such as one or more joints of articulated wrist 224, one or both ends of shaft 310, and/or other joints or links included in articulated arm 222. For example, in some embodiments, rotation of jaws 410 about an axis of symmetry 412 (roll) can be produced by a rotation 414 of shaft 310 about articulated wrist 224. However, in such instances, when rotation 414 of jaws 410 is effectuated by a rotation 414 of shaft 310 about articulated wrist 224, each point of shaft 310 is caused to translate some distance along an arc. Thus, in some instances, translation of portions of articulated wrist 224, shaft 310, and/or other portions of articulated arm 222 occur in conjunction with a commanded rotation of jaws 410 about axis of symmetry 412.

Returning to FIG. 2, a portion of end effector 220 is depicted extending outside of field of view 236 of imaging device 230. In such a situation, the motion of all portions of end effector 220 within worksite 202 and relative to material 204 cannot be easily observed by an operator of computer-assisted system 200 viewing worksite 202 and material 204 via imaging device 230. For example, in some instances, a portion of end effector 220 or articulated arm 222 can extend outside field of view 236 when an operator of computer-assisted system 200 zooms in field of view 236 to confirm that certain material has been captured correctly by end effector 220 and/or a specific instrument associated with a different articulated arm (not shown) is precisely located in the appropriate position and with an appropriate orientation.

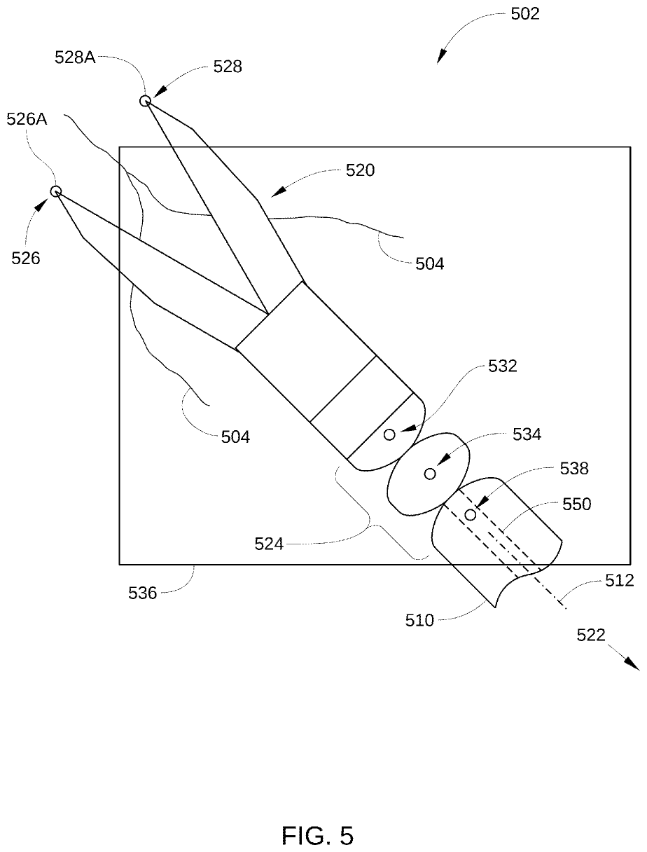

As noted above, certain commanded motions of a control point of end effector 220 (such as a distal portion or tip of end effector 220) can be performed in conjunction with translation of one or more control points included in articulated arm 222. Thus, commanded motions of a control point that is within field of view 236 can result in translation of one or more control points of articulated arm 222 that are outside field of view 236, which is undesirable in many situations. According to various embodiments, translational movement of portions of end effector 220 and/or articulated arm 222 that are not within field of view 236 of imaging device 230 are limited or prevented when commanded motion of control points within field of view 236 is performed. Examples of such embodiments are described below in conjunction with FIGS. 5-7.

FIG. 5 is a simplified diagram showing a portion of an end effector 520 extending outside of a field of view 536 of an imaging device according to some embodiments. For example, end effector 520 can be consistent with end effector 220 of FIG. 2 and field of view 536 can be consistent with field of view 236 of FIG. 2. In the example illustrated in FIG. 5, field of view 536 is depicted in a “camera's eye view,” and therefore shows what is viewable by an imaging device, such as imaging device 230 of FIG. 2. In the example illustrated in FIG. 5, field of view 536 encompasses a portion of a worksite 502, a material 504 (e.g., patient anatomy in a medical example) disposed proximate or within worksite 502, and a portion of end effector 520 disposed within worksite 502. End effector 520 is coupled to an articulated arm 522 by a shaft 510 via an articulated wrist joint 524. Shaft 510 can include various drive mechanisms 550 that are coupled to a drive system (not shown) that can be consistent with drive system 340 in FIG. 3.

According to various embodiments, when control points associated with end effector 520 and/or articulated arm 522 are determined to be disposed outside field of view 536, translation of such control points is prevented when commanded motion of end effector 520 and/or articulated arm 522 otherwise causes such translation. For example, in the instance shown in FIG. 5, a first distal portion 526 and a second distal portion 528 of end effector 520 extend outside of field of view 536, and therefore an operator controlling the motion of end effector 520 cannot view first distal portion 526 or second distal portion 528. In the embodiments, a computer-assisted system that includes end effector 520 and articulated arm 522 determines that a first control point 526A that is associated with first distal portion 526 and a second control point 528A that is associated with second distal portion 528 are disposed outside field of view 536. In response, the computer-assisted system causes first control point 526A and second control point 528A to be in a locked condition. As a result, commands for end effector 520 and/or articulated arm 522 are modified so that the modified commands do not translate first control point 526A and second control point 528A.

In some embodiments, while first control point 526A and second control point 528A are in the locked condition and should not translate as a result of commands, other control points that are associated with end effector 520 and/or articulated arm 522 and are not in the locked condition can be translated by commands. In FIG. 5, examples of such control points include a control point 532 associated and/or co-located with a first rotational joint of articulated wrist joint 524, a control point 534 associated and/or co-located with a second rotational joint of articulated wrist joint 524, and a control point 538 associated and/or co-located with a third rotational joint of articulated wrist joint 524 or an end of shaft 510. Thus, in some examples, translation and/or rotation of control point 532, control point 534, and/or control point 538 is implemented in response to commands provided to a computer-assisted system via input controls, such as input controls 195 in FIG. 1, while translation of first control point 526A and second control point 528A is not implemented in response to such commands. In some examples, such commands are modified so that commanded translation and/or rotation of control point 532, control point 534, and/or control point 538 is adjusted to enable no translation of first control point 526A and second control point 528A. For example, a plurality of joints included in articulated arm 522 can be commanded to move to a first pose or combination of joint positions that cause translation of control point 532 within field of view 536. Upon determining that such commands cause translation of control points that are in a locked condition, such as control point 526A and/or control point 526B, the commands for articulated arm 522 that cause such translation are modified and/or not implemented. When modified, the plurality of joints included in articulated arm 522 can be commanded to move to a second pose or combination of joint positions that causes translation of control point 532 without translation of control point 526A and/or control point 526B.

It is noted that, in a repositionable structure such as articulated arm 522, when commands causing translation of control points in a locked condition are modified so that such translation is not implemented, in some instances, uncommanded translation of such control points can occur. For example, in some instances, uncommanded translation of control points in a locked condition can occur in reaction to commanded motion of other components of the repositionable structure. In another example, in some instances, uncommanded translation of control points in a locked condition can occur in reaction to factors external to the computer-assisted system that includes articulated arm 522, such as motion of worksite 502 relative to articulated arm 522, external force applied to articulated arm 522, and the like. In a medical example, movement of a patient, such as due to breathing, heart beating, and/or the like can cause uncommanded translation of control points in a locked condition. Generally, such uncommanded translation is relatively minor and on the same order as other uncommanded motion of control points that can occur during normal operation of a computer-assisted system.

In some embodiments, control points that are in a locked condition can undergo rotation that is caused by commands input by an operator. In an example, rotation of a control point in a locked condition can be implemented when the rotation is about an axis that passes through the locked control point and does not result in the locked control point (or other locked control points) translating. For instance, in one such example, control point 538, which is associated with an end of shaft 510, can be disposed outside field of view 536, and in such an instance is in a locked condition. In the example, a longitudinal axis 512 of shaft 510 passes through control point 538. Therefore, rotation of control point 538 about longitudinal axis 512 does not result in translation of control point 538 and can occur while control point 538 is in a locked condition. Consequently, in the example, shaft 510 and control point 538 can rotate about longitudinal axis 512 while control point 538 is in the locked condition.

In some embodiments, a control point is in a locked condition when determined to be disposed outside of field of view 536. In some examples, a computer-assisted system determines a control point is outside of field of view 536 based on forward kinematics, computer-vision analysis, and/or manual inputs and/or by a combination of any of these techniques. One of skill in the art will readily understand that any combination of such techniques can be employed for determining whether a control point is outside field of view 536.

In some examples, a computer-assisted system determines that a control point is outside of field of view 536 based on forward kinematics of the imaging device generating field of view 536 and on forward kinematics of articulated arm 522 and/or other joints coupled to end effector 520. In such examples, the position of various control points of articulated arm 522 and/or end effector 520 are determined using forward kinematics of articulated arm 522. For example, the positions of the various control points can be determined in a coordinate frame shared by the imaging device generating field of view 536. Similarly, the location and extent of field of view 536 can be determined using forward kinematics of an articulated arm (not shown) that is associated with the imaging device. For example, the location and extent of field of view 536 can be determined in a coordinate frame shared by articulated arm 522. In the shared coordinate frame, the position of various control points of articulated arm 522 and end effector 520 can be determined relative to the location and extent of field of view 536. In such examples, the position of various control points of articulated arm 522 and/or end effector 520 that are disposed within field of view 536 can be determined when such control points are not visible, such as when occluded by other instruments within field of view 536 or by material 504.

In some examples, a computer-assisted system determines that a control point is outside of field of view 536 based on computer-vision analysis of worksite 502 as viewed by the imaging device generating field of view 536. In such examples, conventional computer-vision algorithms can be employed to identify specific control points of articulated arm 522 and/or end effector 520 that are disposed within field of view 536. Based on the identified control points that are within field of view 536, the computer-assisted system can then determine control points of articulated arm 522 and/or end effector 520 that are disposed outside field of view 536.

In some examples, a computer-assisted system determines that a control point is outside of field of view 536 based on one or more manual user inputs. In such examples, manual user inputs can indicate a specific joint or control point of articulated arm 522 and/or end effector 520 that should be in a locked condition. Thus, in such examples, an operator of a computer-assisted system can cause a control point of articulated arm 522 and/or end effector 520 that is disposed within field of view 536 to be in a locked condition. In some examples, such manual inputs are generated by an operator of the computer-assisted device via input controls of an operator workstation of the computer-assisted device, such as pressing one or more buttons, switches, or pedals of input controls 195 in FIG. 1. In some examples, such manual inputs are generated by an operator via a user interface generated by a display system of the computer-assisted system, such as display system 192 in FIG. 1. For example, the operator can generate a manual input via a touchscreen included in such a display system. In some examples, the operator generates a manual input via a voice command and/or gesture.

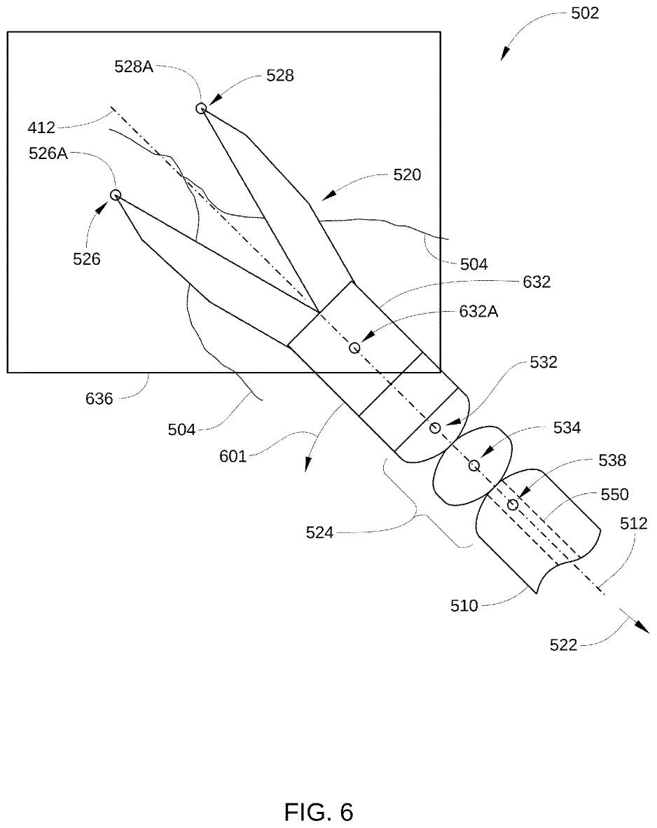

FIG. 6 is a simplified diagram showing end effector 520 disposed within a field of view 636 and coupled to control points outside of field of view 636 according to some embodiments. In the example illustrated in FIG. 6, field of view 636 encompasses a portion of a worksite 502 and material 504 disposed proximate or within worksite 502. As shown, end effector 520 is coupled to one or more control points associated with articulated arm 522 that are disposed outside field of view 636. The control points disposed outside field of view 636 include control point 532, control point 534, and control point 538, which are associated with rotational joints of articulated wrist joint 524 and shaft 510. Therefore, in the example illustrated in FIG. 6, control point 532, control point 534, and control point 538 are in a locked condition and cannot be commanded to translate. By contrast, control points within field of view 636 include first control point 526A associated with first distal portion 526, second control point 528A associated with second distal portion 528, and a control point 632A that is associated with a base portion 632 of end effector 520 are not in a locked condition.

In the example illustrated in FIG. 6, actuation commands for articulated arm 522 that cause motion (rotation and/or translation) of first control point 526A, second control point 528A, and/or control point 632A within field of view 636 and do not cause translation of control point 532, control point 534, or control point 538 can be implemented normally. Thus, actuation commands for articulated arm 522 that can be implemented normally include actuation commands that cause jaws 410 to be opened and/or closed, control point 632A to be translated within field of view 636, and control point 532, control point 534, and/or control point 538 to be rotated about longitudinal axis 512 of shaft 510. In an example, actuation commands for articulated arm 522 that cause a rotation 601 of end effector 520 about control point 532 can be implemented normally until control point 632A is determined to be outside field of view 636. Upon such a determination, actuation commands for articulated arm 522 that cause further rotation of end effector 520 about control point 532 are modified and/or not implemented. By contrast, actuation commands for articulated arm 522 that cause translation control point 532, control point 534, and/or control point 538 to translate are not implemented normally. Instead, such commands can be modified so that translation of control point 532, control point 534, and/or control point 538 does not occur. In an example, actuation commands for articulated arm 522 that cause rotation (roll) of first control point 526A and second control point 528A about axis of symmetry 412 can be implemented normally, while actuation commands for articulated arm 522 that cause translation of control point 532, control point 534, and/or control point 538 are modified and/or not implemented. When modified, various joints included in articulated arm 522 can be commanded to move to a different pose or combination of joint positions than that indicated in the unmodified commands.

In some embodiments, haptic feedback is provided to an operator in response to the operator generating one or more commands for articulated arm 522 that cause translation of one or more control points that are in a locked condition. In an example, such haptic feedback is applied to an input control generating the command that translates the locked control point(s), such as input controls 195 in FIG. 1. In an example, such haptic feedback can include a vibration of a particular input control having a specified magnitude, intensity, and/or duration.

In some embodiments, a perceptual intensity of haptic feedback provided to an operator is related to an actual motion of one or more control points of articulated arm 522 or end effector 520 not matching a commanded motion. Thus, when an operator inputs a commanded motion of articulated arm 522 and/or end effector 520 via an input control, and the commanded motion is modified to avoid the translation of one or more locked control points, haptic feedback is provided to the operator. In an example, the perceptual intensity of haptic feedback provided to the operator is based on an amount by which the actual motion of the one or more control points differs from the commanded motion of the one or more control points. In an example, as the amount by which the actual motion differs from the commanded motion, a magnitude, intensity, and/or duration of the haptic feedback increases. In an example, the perceptual intensity of haptic feedback increases at a rate proportional to the amount the actual motion differs from the commanded motion (e.g., weighted or scaled). In an example, a separate haptic feedback is provided to the operator for each of multiple degrees of freedom. Thus, in such an example, a different perceptual intensity of haptic feedback is provided to the operator for each degree of freedom for which a corresponding actual motion differs from a commanded motion. In another example, a single perceptual intensity of haptic feedback is provided to the operator that is based on a combination of each degree of freedom for which a corresponding actual motion differs from a commanded motion. In such an example, the perceptual intensity of haptic feedback is proportional to a combination of the difference between actual motion and commanded motion for each degree of freedom, such as a vector sum of the differences associated with the various degrees of freedom.

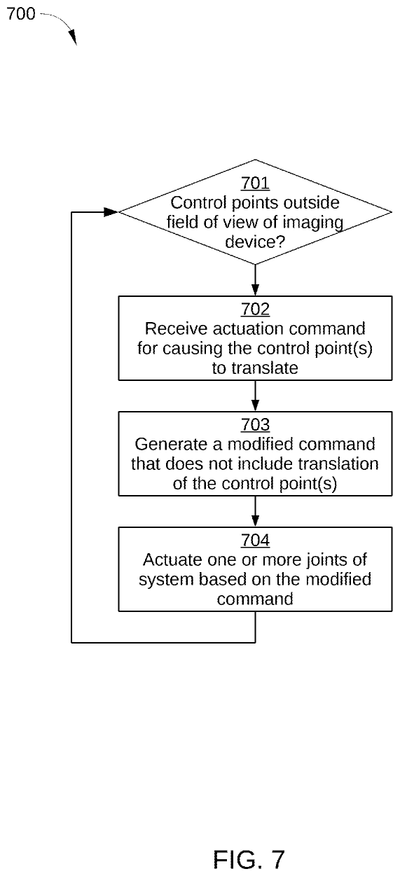

FIG. 7 is a simplified diagram of an exemplary method 700 for local kinematic locking of out-of-view control points according to some embodiments. According to some embodiments, method 700 can include one or more of the processes 701-704, which can be implemented, at least in part, in the form of executable code stored on a non-transitory, tangible, machine readable media that when run on one or more processors (e.g., the processor 140 in control unit 130 of FIG. 1) can cause the one or more processors to perform one or more of the processes 701-704.

At process 701, the determination is made whether there are any control points for a repositionable structure, such as articulated arm 522 in FIG. 5, that are disposed outside a field of view of an imaging device associated with a computer-assisted system. As described above, such a determination can be made based on forward kinematics, computer-vision analysis, and/or one or more manual inputs that are performed by an operator of the computer-assisted system and/or any combination of these techniques. In an example, the one or more control points determined to be disposed outside the field of view of the imaging device are placed in a locked condition. In such an example, commands causing translation of the control points in the locked condition can be modified so that such translation is not implemented, as described below.

At process 702, one or more actuation commands are received by the computer-assisted system for causing at least one of the control points in the locked condition to translate. In an example, the one or more actuation commands can be received via operator inputs, such as by the manipulation of one of input controls 195.

At process 703, one or more modified commands are generated that are based on the one or more actuation commands received in process 702. In an example, the modified commands do not cause translation of one or more control points in the locked condition. In an example, the one or more modified commands are generated via modification of the one or more actuation commands.

At process 704, the one or more modified commands are employed by the computer-assisted system. In an example, the one or more modified commands cause one or more joints of the computer-assisted system to be actuated, where such actuation does not result in translation of the one or more control points that are in the locked condition. In another example, the one or more modified commands prevent one or more joints of the computer-assisted system from being actuated, so that translation of the one or more control points in the locked condition do not translate. In an example, haptic feedback is generated in process 704 when the modified commands are employed. Upon completion of process 704, method 700 returns to process 701.

Although illustrative embodiments have been shown and described, a wide range of modification, change and substitution is contemplated in the foregoing disclosure and in some instances, some features of the embodiments may be employed without a corresponding use of other features. One of ordinary skill in the art would recognize many variations, alternatives, and modifications. Thus, the scope of the invention should be limited only by the following claims, and it is appropriate that the claims be construed broadly and, in a manner, consistent with the scope of the embodiments disclosed herein.

Any and all combinations of any of the claim elements recited in any of the claims and/or any elements described in this application, in any fashion, fall within the contemplated scope of the present invention and protection.

The descriptions of the various embodiments have been presented for purposes of illustration, but are not intended to be exhaustive or limited to the embodiments disclosed. Many modifications and variations will be apparent to those of ordinary skill in the art without departing from the scope and spirit of the described embodiments.

Aspects of the present embodiments may be embodied as a system, method or computer program product. Accordingly, aspects of the present disclosure may take the form of an entirely hardware embodiment, an entirely software embodiment (including firmware, resident software, micro-code, etc.) or an embodiment combining software and hardware aspects that may all generally be referred to herein as a “module,” a “system,” or a “computer.” In addition, any hardware and/or software technique, process, function, component, engine, module, or system described in the present disclosure may be implemented as a circuit or set of circuits. Furthermore, aspects of the present disclosure may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

Aspects of the present disclosure are described above with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine. The instructions, when executed via the processor of the computer or other programmable data processing apparatus, enable the implementation of the functions/acts specified in the flowchart and/or block diagram block or blocks. Such processors may be, without limitation, general purpose processors, special-purpose processors, application-specific processors, or field-programmable gate arrays.

The flowchart and block diagrams in the figures illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

While the preceding is directed to embodiments of the present disclosure, other and further embodiments of the disclosure may be devised without departing from the basic scope thereof, and the scope thereof is determined by the claims that follow.

1. A computer-assisted system comprising:
a repositionable structure that includes one or more joints coupled to an end effector; and
a control unit,
wherein the control unit is configured to:
determine that a control point associated with the end effector is not within a field of view of an imaging device capturing images of a worksite;
while the control point is not within the field of view of the imaging device, receive an actuation command for causing the control point to translate;
based on the actuation command, generate a modified command that does not translate the control point; and
actuate one or more joints of the repositionable structure or the end effector based on the modified command.

2. The computer-assisted system of claim 1, wherein the modified command rotates the control point.

3. The computer-assisted system of claim 1, wherein the control point is included in the end effector.

4. The computer-assisted system of claim 1, wherein the control point corresponds to a joint of the end effector.

5. The computer-assisted system of claim 1, wherein the control point corresponds to a distal portion of the end effector.

6. (canceled)

7. The computer-assisted system of claim 1, wherein the actuation command causes one or more of a stapling operation, a grasping operation, a cutting operation, an energy delivery operation, a translation of at least a portion of the end effector, or a rotation of at least a portion of the end effector.

8-9. (canceled)

10. The computer-assisted system of claim 1, wherein the control unit further applies haptic feedback on an input control of the computer-assisted system.

11. (canceled)

12. The computer-assisted system of claim 1, wherein the end effector includes one of a surgical stapler, a suction irrigator, an electrocautery device, a gripper, or a cutting mechanism.

13. The computer-assisted system of claim 1, wherein to determine that the control point is not within the field of view of the imaging device, the control unit is configured to determine a current location of the control point based on forward kinematics of the repositionable structure.

14. The computer-assisted system of claim 1, wherein to determine that the control point is not within the field of view of the imaging device, the control unit is configured to receive a user input indicating the control point.

15. The computer-assisted system of claim 1, wherein to determine that the control point is not within the field of view of the imaging device, the control unit is configured to perform computer-vision analysis of the field of view based on information generated by the imaging device.

16. The computer-assisted system of claim 1, wherein to determine that the control point is not within the field of view of the imaging device, the control unit is configured to utilize two or more of determining a current location of the control point based on forward kinematics of the repositionable structure, receiving a user input indicating the control point, or performing computer-vision analysis of the field of view based on information generated by the imaging device.

17. (canceled)

18. A method for operating a computer-assisted device, the computer-assisted device comprising a repositionable structure that includes one or more joints coupled to an end effector, the method comprising:
determining, by a control unit, that a control point associated with the end effector is not within a field of view of an imaging device capturing images of a worksite;
while the control point is not within the field of view of the imaging device, receiving, by the control unit, an actuation command for causing the control point to translate;
based on the actuation command, generating, by the control unit, a modified command that does not translate the control point; and
actuating, by the control unit using one or more motors, solenoids, servos, or actuators, one or more joints of the repositionable structure or the end effector based on the modified command.

19. The method of claim 18, wherein the modified command rotates the control point.

20. The method of claim 18, wherein:
the control point is included in the end effector;
the control point corresponds to a joint of the end effector; or
the control point corresponds to a distal portion of the end effector.

21-26. (canceled)

27. The method of claim 18, further comprising applying haptic feedback on an input control.

28-32. (canceled)

33. The method of claim 18, wherein determining that the control point is not within the field of view of the imaging device comprises two or more of determining a current location of the control point based on forward kinematics of the repositionable structure, receiving a user input indicating the control point, or performing computer-vision analysis of the field of view based on information generated by the imaging device.

34. (canceled)

35. A non-transitory machine-readable medium comprising a plurality of machine-readable instructions which when executed by one or more processors associated with a computer-assisted device, are adapted to cause the computer-assisted device to perform a method for operating the computer-assisted device, the computer-assisted device comprising a repositionable structure that includes one or more joints coupled to an end effector, the method comprising:
determining that a control point associated with the end effector is not within a field of view of an imaging device capturing images of a worksite;
while the control point is not within the field of view of the imaging device, receiving an actuation command for causing the control point to translate;
based on the actuation command, generating a modified command that does not translate the control point; and
actuating, using one or more motors, solenoids, servos, or actuators, one or more joints of the repositionable structure or the end effector based on the modified command.

36. The non-transitory machine-readable medium of claim 35, wherein the modified command rotates the control point.

37. The non-transitory machine-readable medium of claim 35, wherein:
the control point is included in the end effector;
the control point corresponds to a joint of the end effector; or
the control point corresponds to a distal portion of the end effector.
